(19) United States
(12) Patent Application Publication
(10) Pub. No.: US 2006/0137619 A1
(43) Pub. Date: Jun. 29, 2006

(54) Automatic freshener system and method

(76) Inventors: Daniel N. Dodman, Westborough, MA (US); Nicholas H. Dodman, Westborough, MA (US)

Correspondence Address: Brian M. Dingman, Esq.;Mirick, O'Connell, DeMallie & Lougee, LLP, 1700 West Park Drive, Westborough, MA, 01581-3941, US

(21) Appl. No.: 11/319,690

(22) Filed: Dec. 28, 2005

Related U.S. Application Data
(60) Provisional application No. 60/640,037, filed on Dec. 29, 2004.

(51) Int. Cl. A01K 1/035

(52) U.S. Cl. 119/163

(57) ABSTRACT

A system and method for automatically applying an odor neutralizing or masking agent or a pheromonal agent to a cat litter box, or an odor neutralizing or masking agent to an area around a human toilet. There is a source of spray delivery of the agent, and a motion sensor. The spray delivery source applies the agent in response to the motion sensor, so that the agent is applied only after the cat or human has finished using the facility.

### CROSS REFERENCE TO RELATED APPLICATION

This application claims priority of provisional application Ser. No. 60/640,037, filed on Dec. 29, 2004.

### FIELD OF THE INVENTION

The present invention is concerned with reducing cat litter box odors for the benefit of cat owners and cats alike, as well as with automatic odor reduction in human toilets.

### BACKGROUND OF THE INVENTION

Since the fortuitous discovery by Edward Lowe, in Michigan in 1947, that cats would naturally gravitate to using kiln-dried granulated clay in litter pans to perform their excretory functions (urination and defecation), keeping cats indoors has become a much more common practice and is safer for the cat. It is estimated that approximately half of the nation's 70 million cats are “indoor only” cats and the average life span of domestic cats, so kept, has increased dramatically as a result since they no longer have to be exposed to the perils of outdoor life, including trauma and disease. There is, however, a down side to this seemingly Utopian situation in that cat owners are often bothered by the odor of their cat's litter boxes and take measures to address their concern that are not optimal for the cat's continuing use of these designated excretory facilities. Some owners, for example, try reducing the number of litter boxes in the house to limit the number of olfactorally-contaminated areas. Others try relegating the litter boxes to out of the way areas such as closets or cellars. The result of these interventions is often to shift the cat's attention to other areas for elimination and frequent substrates chosen for this purpose by the cat are throw rugs, carpets, clothing, and even furniture. In fact, what is euphemistically referred to as “inappropriate elimination”—failure to use the litter boxes provided—is the number one behavior problem reported by owners of domestic cats and affects a large percentage of owners at some point or other during their cat's life. While some owners might seek professional guidance, others seek their own solutions and often fail, eventually relinquishing their charges to the nearest shelter or pound. Two of three cats surrendered to shelters and pounds are euthanatized, so indirectly this problem is responsible for more cat deaths than any other medical or behavioral condition. For owners who refuse to surrender their cats, living with their problem eliminator creates an anti-social and unhygienic situation with attenuation of the so-called human-companion animal bond. The problem continues to be a great source of stress for these owners and has led to thousands of dollars worth of expense due to destroyed property (urine-soaked carpets and rugs), matrimonial discord, divorce, and even the serial sale of homes. This whole problem starts because of the unacceptable odor emanating from a used litter box and translates down the line to expense and mental suffering for owners and often times the death of a cat.

Litter companies have attempted to address this weighty problem by adding various aromatic or odor-reducing particles to the litter. Various additives have been used ranging from baking soda to carbon particles and various perfumes. None has been entirely successful and many litter additives, while perhaps producing some marginal benefit for owners, have actually served as a further deterrent for cats. It is generally believed by behaviorists that cats do not appreciate chemical or floral essences in litters and may even be repelled from the litter box by them. In other words, the partial solution to the owner's problem can create an even more serious and potentially deadly problem from the cat, that of inappropriate elimination.

When dealing with the problem of inappropriate elimination, it is common practice to invoke a yin and yang-type approach whereby litter boxes are cleaned more frequently and generally made more attractive while using odor neutralizing chemicals (not perfumed or other masking odors) to treat soiled areas around the house. What has not really been exploited is the use of odor-neutralizing products, like enzymes, live bacterial preparations, or odorlyzing chemicals to treat the litter box itself. This would have the dual benefit of improving the atmosphere around the litter box for the benefit of the owner but also making the litter box more attractive for the cat because cats are also repelled by litter box odors. One company, Zero Odor LLC, has more recently started to advertise their product for freshening the litter box in this way though the use of other odor neutralizing products would also be encompassed by this general concept. One drawback to the practice of freshening the litter box in this way is that an owner cannot always be present when the cat uses the litter box facility provided, giving time for odors to permeate the environment, and until subsequent scooping and odor neutralization of the box leaving somewhat of a deterrent for a cat visiting the box meanwhile. Even if the box is religiously sprayed two or three times a day there is still plenty of opportunity to miss cats litter box visits, particularly at night or during owner absences. The present invention serves to address this problem.

### SUMMARY OF THE INVENTION

The present invention provides for the automatic dispensation of an odor-eliminating or odor-neutralizing product to cats' litter boxes—or indeed to a human toilet facility—following use of this facility. In this way, the atmosphere around the litter box or toilet is freshened immediately following each use, making the ambient air non-offensive and increasing the attractiveness of the facility for subsequent users.

### BRIEF DESCRIPTION OF THE DRAWINGS

Other objects, features and advantages will occur to those skilled in the art from the following description of the preferred embodiments and the accompanying drawings, in which:

FIG. 1A schematically depicts a stand-alone version of a system according to the invention for automatically neutralizing or masking odor from a cat litter box, or for pheromonal treatment of a litter box, or for freshening an area around a toilet. The system also accomplishes the inventive method;

FIG. 1B is a side view of the system of FIG. 1A;

FIG. 2A is a top view of a litter box or toilet stall with the inventive system incorporated therein;

FIG. 2B is a side view of the box or stall of FIG. 2A; and

FIG. 3 is a flow chart of the operation of the preferred embodiment of the method of the invention.

### DETAILED DESCRIPTION OF THE INVENTION

The invention comprises a device, a container of some sort, be it canister, bottle or some other sealed container that contains an odor eliminating or odor neutralizing product, either in concentrated form, as gas or liquid, and/or dissolved/suspended in some vehicle. The material contained within the container is released automatically within a short time of the cat or person exiting the litter box or toilet facility. This can be achieved by having a motion-sensing device, integral or separate from the container, which activates a delay circuit to open a valve releasing the contained material in a burst or spray. Clearly this would be counterproductive if it occurred while the subject was engaged in using the facility, so the motion-sensor, while actually sensing motion in the immediate vicinity, would transmit a signal that would keep the valve shut. When the sensing signal was lost—i.e. immediately after the subject had left the facility—a delay circuit would be activated, to release the enzyme or chemical containing spray within seconds or minutes of the subject's departure from the facility.

In one embodiment of the invention, the enzymatic or chemical-containing container is an aerosol can in which the contents are under pressure. The spray device, motion sensor, and electronic circuitry are contained within a plastic housing that can be attached on top of the aforesaid aerosol spray can. This aerosol and controlled-release device combination would be placed close to the cats' litter box perhaps some 12 to 18 inches from the side of the box and is directed toward the center of the box. However, it is also envisaged that the aerosol device might be affixed to a wall close to the box and/or suspended and angulated toward the box.

In another embodiment of the invention, the liquid or other pressurized enzyme or chemical containing material would be in a container that is integral with the litter box, as would the motion-sensing device delay circuit and valve control spray port.

Yet another embodiment of the invention, the canister, be it an aerosol canister or other container, with its associated motion-sensor electronic circuitry and valve control spray mechanism or nuzzle would be positioned close to a human toilet and angled suitably and at such a distance from the toilet that the spray would disperse evenly in such a way as to envelop all odor causing particles or gases.

A fourth embodiment of the invention would be with the same canister motion-sensor, electronic circuitry, and valve-controlled spray device, integral with the toilet tank or any other fixed component of the toilet itself, be that seat or bowl.

It is envisaged that the container in any of the above embodiments the invention would be made of metal or plastic or glass. It is also envisaged that the control device for the spray, including the motion-sensor and circuitry, be contained in a unit that is either non-disposable or disposable. Furthermore, it is envisaged that the material vaporized or volatilized under pressure or force be comprised of one of the following:
a) Preferably the material known by the trade name of Zero Odor™ (Zero Odor LLC, Pound Ridge, N.Y.) as its action is absolute in the sense that the molecules of the chemical contained in Zero Odor™ combine irreversibly with odor-causing molecules to precipitate them and/or inentivate them, thus removing odors in their entirety. This has been demonstrated quite conclusively in cat litter box experiments where the gases above the litter surface have been found to comprise of a dozen or more self-containing amino acids. These peaks can be detected on a sensitive gas chromatograph. Following treatment of the box with Zero Odor™, these peaks are no longer detectible and to the sense of smell the odor is gone. A similar level of efficacy in combating odors would be anticipated when dealing with odors emanating from human toilet facilities.
b) Another possibility for the active component contained within the spray device might be an as-yet unmarketed or undiscovered chemical that combines with, for instance, methyl groups, sulphur or sulphide bonds, or acts as an oxidizing or reducing agent, in such a way as to render the chemical or particulate matter that causes odors innocuous or inoffensive.
c) Another possibility for the liquid contained within the automatic spray dispenser would be an enzymatic preparation which, overtime, would break down the molecules causing odors. While potentially effective this would not be the preferred method of treatment as the breakdown of odor-controlling chemicals by enzymes takes some appreciable time and preferably the odor neutralization would be immediate.
d) A fourth possibility for the material contained within the spray device would be a solution containing live bacteria or other biological agent that was effective at breaking down molecules odors yet, at the same time, was intrinsically innocuous to man and other animals (i.e. non-pathogenic).
e) A fifth possibility for the fluid or gas contained within the spray container might be some masking odor demonstrated not to be offensive to cats in any concentration appreciable by them or humans. Specifically excluded from the invention for use in litter boxes would be fluids or gases that are aversive to cats. While masking odors is not the preferred method of the invention, it is nevertheless encompassed by it.
f) A sixth and final potential composition to be dispensed by the automatic spray device would be any combination of the five options listed above. For example, a combination of Zero Odor™ plus a trace of masking odor that was inoffensive to cats.

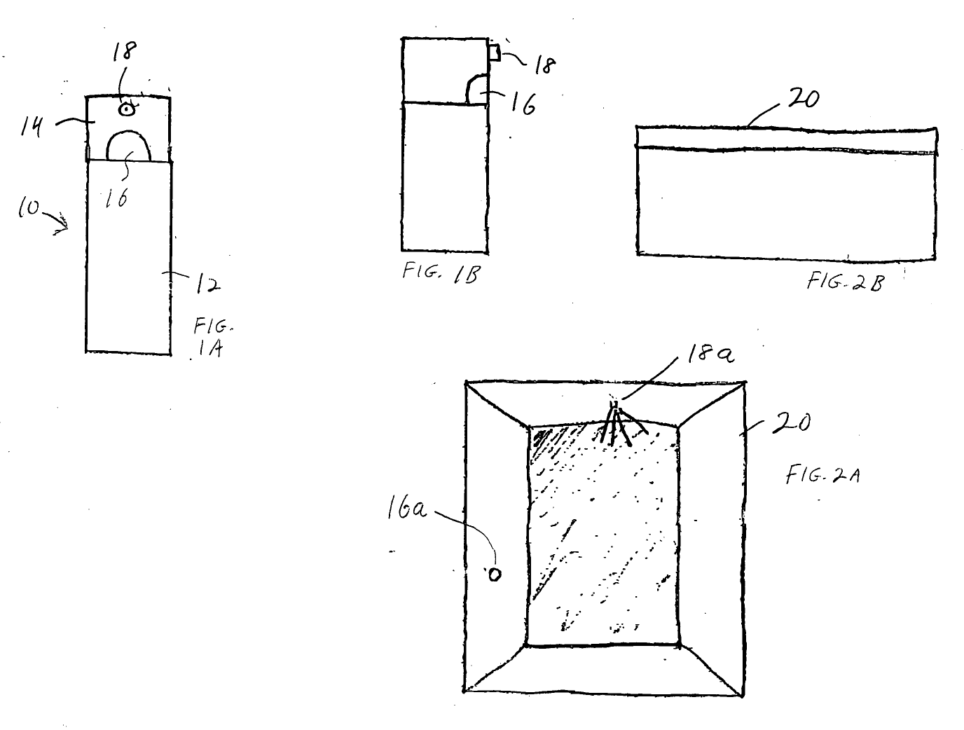

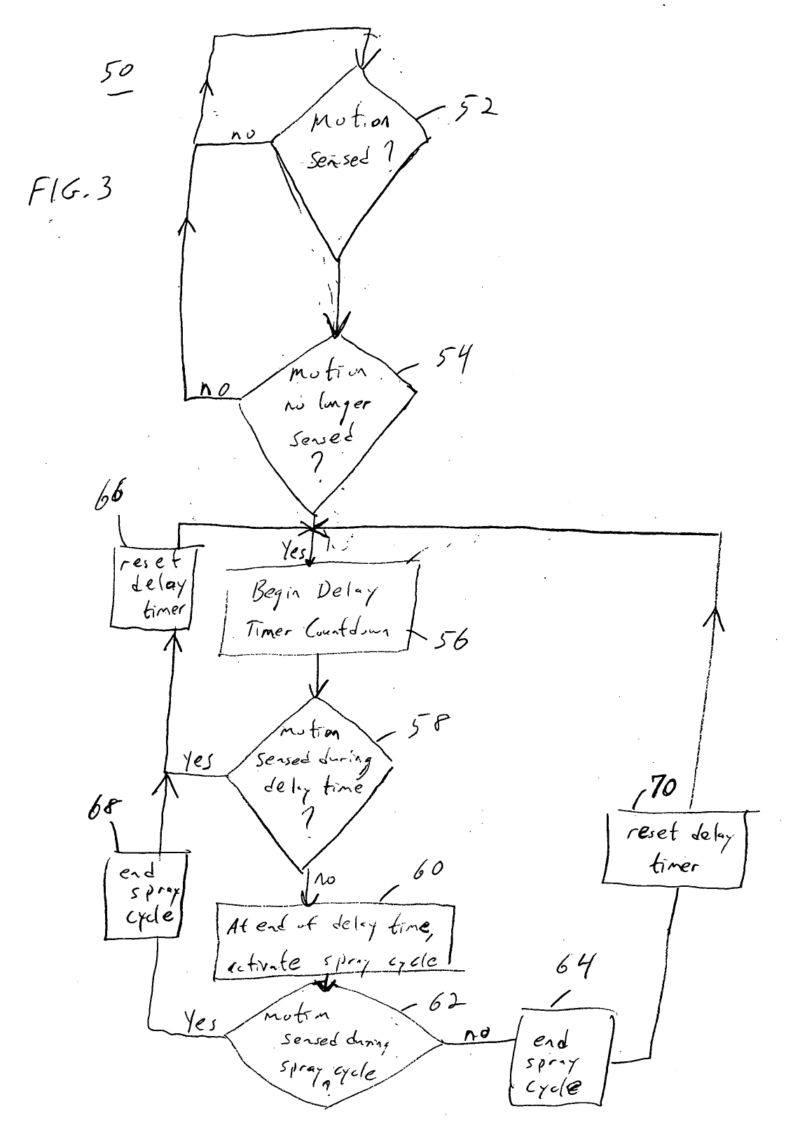

Some potential arrangements for the automatic litter spray for cats are shown in FIGS. 1 and 2. A flow chart of its operation is shown in FIG. 3. The human toilet application of the device is essentially the same as described above for the cat product and would be organized as described above.

The invention comprises a motion-sensitive automatic freshening spray system and method. One embodiment is designed to olfactorily freshen or modify cats' litter with one or more beneficial substances that are sprayed into the litter after every visit a cat makes to the box. A component is a delay circuit that prevents the cat from being sprayed while it is in the box. The presence of the cat in the box will suppress activation of the spray, which may be activated some time (typically 30 seconds to 5 minutes) after the cat has left the box. If the cat approaches the box again within this time period, the mechanism will reset so that the cat is not sprayed. The spray can be activated briefly, perhaps delivering single or multiple 1-5 second bursts, and then become inactive until reactivated by the presence of a cat. Physically, the motion-sensitive, timed-delivery device could be a stand alone unit or it could be incorporated into the litter box itself. The stand alone unit might take the form of an aerosol canister with the sensor and spray control device atop and it may rest on the floor. Alternatively, it may hang above the box or be mounted beside the box. The method of delivery of the spray is not limited to a pressurized canister approach and may take the form of, say, a piston pump or other form of spray delivery.

In one usage of this embodiment, a number of different odor-neutralizing or odor-masking compounds may be used. Preferably, the active ingredient would be an oxidizing agent (or other chemical), enzyme(s), or biological agent. In another usage of this embodiment, other compounds that may improve the litter box appeal, including but not limited to pheromones/pheromonal attractants, may be applied to the litter box.

Currently, immediate motion-sensitive deterrents and delayed response mechanical litter box cleaners exist; the motion sensing, timing and delivery circuits are all known per se and so are not disclosed per se herein. Also, odor neutralization and odor masking products are made. Pheromonal treatment for litter box problems is also known. What distinguishes this embodiment of the invention is that it accomplishes motion-sensitive, delayed delivery of a product for the litter box using appropriate compounds administered automatically and in a timely fashion. The whole concept of applying continuous litter box hygiene in this manner (as opposed to, say, using perfumed or carbon granule-impregnated cat litter) is entirely new and this device enables it.

In another embodiment of the invention, an odor neutralization or odor masking product can be automatically spray applied to an area around a human toilet. The same system and methodology can be used to automatically spray apply an appropriate odor neutralizer to freshen the location for the next user. Both embodiments disclosed below are appropriate for such human usage.

FIGS. 1A and 1B schematically depict a stand-alone version of system 10 for automatically neutralizing or masking odor from a cat litter box, or for pheromonal treatment of a litter box, or for freshening an area around a toilet. System 10 includes a source 12 of spray delivery of an appropriate agent, in this embodiment accomplished with a spray can 12 containing the agent. Detachable top portion 14 comprises motion sensor 16 that detects motion in litter box (or toilet area) 20, and spray nozzle 18 that directs spray from can 12 toward litter box (or toilet area) 20. The agent reservoir and spray system can be mounted on a wall. Timed spray applicator devices are known in the deodorizing field, as for example are commonly used in rest rooms.

In the version of the invention shown in FIGS. 2A and 2B that is incorporated into the wall of a litter box or the wall of a toilet stall, sensor 16a is preferably located on one of the vertical walls and spray nozzle 18a located on the same or a different wall. For covered litter boxes, one or both of these devices may be located on the cover.

A basic flow chart explaining the methodology of the invention is shown in FIG. 3, in which flow chart 50 begins with step 52 in which motion is sensed by sensor 16. When motion is sensed, the system processor periodically queries the sensor to determine whether motion is still sensed, step 54. This query time would be set as desired to achieve the result. When motion was no longer sensed, a delayed timer countdown in the processor would begin, step 56. If motion was sensed during the delay time, step 58, the delay timer would be reset, step 66, and operation would proceed to motion sensing step 52. If motion was not sensed during the delay time, at the end of the delay time the particular spray cycle would be activated, step 60. if motion was sensed during the spray cycle, step 62, the spray cycle would be ended, step 68, and the delay timer would be reset, step 66, and operation would return to step 52. If motion was not sensed during the spray cycle, the spray cycle would continue its course until its end time, step 64, after which the delay timer would be reset, step 70, and operation returned to first step 52. In the preferred embodiment, the query rate of the motion sensor, the delay time, and the spray cycle would be established in software or firmware. However, for more sophisticated uses, the delay timer and/or the particular spray cycle could be set by the user (or perhaps the distributor before installation in a toilet area) from a menu of options stored in the memory of the system.

Important components of the invention are the motion-sensor, the electronic circuitry containing cutoff (for when the litter box or toilet facility is occupied), the delay circuit, the liquid or gas containing vessel, and the valve-controlled spray nozzle, plus the odor neutralizing eliminating or masking chemicals or biological products described above. The invention covers all these components as an entity and not any one in isolation.

Although specific features of the invention are shown in some drawings and not others, this is for convenience only as the features may be combined in other fashions in accordance with the invention.

Other embodiments will occur to those skilled in the art and are within the following claims.

What is claimed is:

1. A system for automatically applying an odor neutralizing or masking agent or a pheromonal agent to a cat litter box, or an odor neutralizing or masking agent to an area around a human toilet, comprising:
a source of spray delivery of the agent;
a motion sensor;
a delay circuit; and
means, responsive to the motion sensor and the delay circuit, for causing the spray delivery source to apply the agent.

2. The system of claim 1, wherein the means for causing application of the agent comprises means for delaying application for a delay time that counts down after motion is no longer sensed.

3. A method of automatically applying an odor neutralizing or masking agent or a pheromonal agent to a cat litter box, or an odor neutralizing or masking agent to an area around a human toilet, comprising:
providing a source of spray delivery of the agent;
providing a motion sensor;
providing a delay; and
in response to the motion sensor, and after a delay once there is no longer motion sensed, causing the spray delivery source to apply the agent.
